(19) United States
(12) Patent Application Publication
(10) Pub. No.: US 2024/0352482 A1
(43) Pub. Date: Oct. 24, 2024

(54) GENETICALLY MODIFIED HIGH PRODUCTIVITY INSECT CELL LINES AND METHODS FOR OBTAINING SAME

(71) Applicant: Virovek, Inc., Hayward, CA (US)

(72) Inventor: Haifeng Chen, Piedmont, CA (US)

(21) Appl. No.: 18/414,293

(22) Filed: Jan. 16, 2024

Related U.S. Application Data
(60) Provisional application No. 63/475,541, filed on Nov. 16, 2022.

(51) Int. Cl. C12N 15/86; C12N 5/07

(52) U.S. Cl. CPC C12N 15/86; C12N 5/0601; C12N 2710/14022; C12N 2710/14044; C12N 2750/14143; C12N 2750/14152

(57) ABSTRACT

The present disclosure provides a novel genetically modified high productivity VVK-V432C insect cell line derived from the ATCC CRL-1711 cell line. Methods for obtaining this high productivity cell line are also provided.

### CROSS REFERENCE TO RELATED APPLICATIONS

This application claims the benefit of U.S. Provisional Patent Application No. 63/475,541 filed Nov. 16, 2022, the entire contents of which are hereby incorporated by reference.

### SEQUENCE LISTING

The instant application contains a Sequence Listing which has been submitted electronically in XML file format and is hereby incorporated by reference in its entirety. Said XML copy, created on Jun. 26, 2024, is named 2014202-0021_SL.xml and is 9,107 bytes in size.

### FIELD

The current teachings generally relate to high productivity insect cell lines that are used for AAV production and protein expression. The current teachings also relate to methods for obtaining the high productivity cell lines that are derived from cells or organisms that are low productivity for AAV production and protein expression.

### BACKGROUND

Since it was first described in the peer-reviewed literature in the early 1980's, the baculovirus-insect cell system (BICS) has become a widely recognized and heavily utilized recombinant protein production platform. The insect cell lines most commonly used as hosts in the BICS are derived from the cabbage looper, Trichoplusia ni (Tn), or fall armyworm, Spodoptera frugiperda (Sf), and most biologics manufactured with the BICS are produced using the latter. However, there exists a need for cell lines that can be maintained at high-passage yet retain high productivity and for methods to generate such cell lines.

### SUMMARY

The present disclosure relates to genetically modified cell lines derived from cells or organisms. In some embodiments, such cell lines are characterized by an increase of AAV vector productivity and/or protein expression levels. Such established cell lines are particularly useful as components of biological platforms used for production of AAV vectors, vaccines, recombinant proteins and biologics for human and veterinary use. The present disclosure also relates to methods for obtaining such genetically modified cell lines from cells or organisms that are less productive.

In one aspect, the disclosure features an insect cell line (e.g., a genetically modified insect cell line) comprising a nucleotide sequence encoding baculovirus IEL. In some embodiments, the nucleotide sequence is or comprises a nucleotide sequence described herein. In some embodiments, the cell line constitutively expresses baculovirus IE1.

In some embodiments, the cell line is characterized by a shorter doubling time (e.g., by about 1 hour, 2, hours, 3 hours, 4 hours, 5 hours, 6 hours, 7 hours, 8 hours, 9 hours, 10 hours, 12 hours, 18 hours, 24 hours) relative to a parental cell line, when propagated under the same conditions. In some embodiments, the cell line is characterized by higher AAV production yield (e.g., by about 10%, 20%, 30%, 40%, 50%, 60%, 70%, 80%, 90%, 100%, or more) relative to a parental cell line.

In some embodiments, the cell line is derived from an insect. In some embodiments, the cell line is derived from a lepidopteran insect. In some embodiments, the lepidopteran insect comprises Spodoptera frugiperda, Trichoplusia ni, or Bombyx mori.

In some embodiments, the cell line is derived from a Trichoplusia ni cell line. In some embodiments, the cell line is derived from a Bombyx mori cell line. In some embodiments, the cell line is derived from a Spodoptera frugiperda cell line.

In some embodiments, the cell line is derived from a Spodoptera frugiperda cell line. In some embodiments, the cell line is derived from Spodoptera frugiperda Sf-9, e.g., designated ATCC CRL-1711. In some embodiments, the cell line is characterized by a shorter doubling time (e.g., by about 1 hour, 2, hours, 3 hours, 4 hours, 5 hours, 6 hours) relative to parental cell line Spodoptera frugiperda Sf-9 designated ATCC CRL-1711, when propagated under the same conditions. In some embodiments, the cell line is characterized by higher AAV production yield (e.g., by about 10%, 20%, 30%, 40%, 50%, 60%, 70%, 80%, 90%, 100%, or more) relative to parental cell line Spodoptera frugiperda Sf-9 designated ATCC CRL-1711.

In another aspect, the disclosure provides a method of generating a recombinant baculovirus (rBV). In some embodiments, the method includes (a) culturing an insect cell line (e.g., a genetically modified insect cell line) comprising (i) a nucleotide sequence encoding baculovirus IE1 and (ii) one or more nucleic acids encoding a protein for production of a recombinant baculovirus (rBV), and (b) harvesting the cell culture. In some embodiments, the insect cell line (e.g., a genetically modified insect cell line) are cultured under conditions sufficient to produce a recombinant baculovirus (rBV).

In another aspect, the disclosure provides a method of producing AAV vectors. In some embodiments, the method includes (a) infecting an insect cell line (e.g., a genetically modified insect cell line) comprising a nucleotide sequence encoding baculovirus IE1 with a recombinant baculovirus (rBV) comprising at least one foreign protein gene; (b) culturing the infected cell under conditions conducive for the expression of the foreign protein gene; and (c) isolating the foreign protein. In some embodiments, the foreign protein gene comprises at least one viral protein gene and/or at least one mammalian protein gene. In some embodiments, the viral protein gene comprises a gene that encodes an AAV protein, an adenoviral protein, a retroviral protein, an SV40 protein, or a Herpes simplex viral protein.

### BRIEF DESCRIPTION OF THE DRAWINGS

FIG. 1 is a schematic map of plasmid V432-pcDNA-CMV-IE1-pIE1-NeoR_KanR. IE1 is under the control of CMV promoter and SV40 polyadenylation signal. The NeoR_KanR resistant gene is under control of the IE1 promoter and a second SV40 polyadenylation signal.

FIGS. 2A-2F show five non-limiting examples of AAV vector production and one non-limiting example of protein expression using the VVK-V432C cell line. FIG. 2A depicts an exemplary single infection of VVK-V432C cell line by one rBV carrying AAV rep-cap genes and a GOI flanked by AAV ITRs to produce AAV vectors. FIG. 2B depicts exemplary dual infection of VVK-V432C cell line by a first rBV carrying AAV rep-cap genes and a second rBV carrying a GOI flanked by the AAV ITRs to produce AAV vectors. FIG. 2C depicts exemplary single infection of VVK-V432C cell line stably transfected with AAV-rep genes by one rBV carrying AAV-cap and a GOI flanked by the AAV ITRs to produce AAV vectors. FIG. 2D depicts exemplary single infection of VVK-V432C cell line stably transfected with AAV-rep-cap genes by one rBV carrying a GOI flanked by the AAV ITRs to produce AAV vectors. FIG. 2E depicts exemplary single infection of VVK-V432C cell line stably transfected with a GOI flanked by the AAV ITRs by one rBV carrying the AAV rep-cap genes to produce AAV vectors.

FIG. 2F depicts exemplary single infection of VVK-V432C cell line by one rBV carrying a Target Gene to express the target protein.

FIG. 3 is an agarose DNA gel image showing the amplification of the IE1 gene from VVK-V432 clone A and clone C cells.

FIG. 4 is an SDS-PAGE gel image showing the that a variety of serotypes of AAV vectors produced in the VVK-V432C cell line have the correct VP1, VP2, and VP3 ratios. M, protein ladder; lane 1, control AAV9 vector from Sf9 cells; lanes 2 & 3, AAV5 vectors from VVK-V432A (ESF921 media); lanes 4 & 5, AAV8 vector from VVK-V432A (ESF921 media); lanes 6 & 7, AAV8 vector from VVK-V432C (ESF921); lanes 8 & 9, AAV8 vector from VVK-V432A (ESFAF media); lanes 10 & 11, AAV8 vector from VVK-V432C (ESFAF media).

FIG. 5 shows GFP expression intensity comparisons between 4 different dilutions of AAV transductions control HEK293 cells without AAV transduction, AAV vectors produced in the genetically modified cell line CCK-V432C, and AAV vectors produced in the control Sf9 cell lines.

### DEFINITIONS

In order for the present invention to be more readily understood, certain terms are first defined below. Additional definitions for the following terms and other terms are set forth throughout the specification.

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

The term “cell line”, when used herein, means a population of cells that are expanded from one or more common cells (e.g., ancestor cell, parent cell), for example but not limited to, a clonal population of cells that have been expanded from a single isolated cell. An “established cell line”, as used herein, is a cell line that has the potential to proliferate indefinitely when given fresh culture media, space to grow, and when incubated under suitable conditions. Such cell lines have undergone changes in vitro (for example but not limited to transformation, chromosomal changes, or both) compared to the naturally-occurring counterpart cell found in the organism. A cell line that is obtained by isolating a single cell from a first cell line, then expanding the isolated cell to obtain a multiplicity of cells to obtain a second cell line, is sometimes referred to as a “subclone” of the first cell line from which it was derived.

The term “encode” or “encoding” refers to sequence information of a first molecule that guides production of a second molecule having a defined sequence of nucleotides or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or an RNA molecule encodes a polypeptide if transcription and translation of RNA corresponding to that gene produces the polypeptide in a cell or other biological system.

The term “expression” of a nucleic acid sequence refers to the generation of a gene product from the nucleic acid sequence. In some embodiments, a gene product can be a transcript, e.g., a polyribonucleotide as provided herein. In some embodiments, a gene product can be a polypeptide.

As used herein, the term “comprising”, which is synonymous with “including” or “characterized by”, and cognates of each (such as comprises and includes), is inclusive or open-ended and does not exclude additional unrecited components, elements, or method steps, that is other components, steps, etc., are optionally present. For example, but not limited to, an article “comprising” components A, B, and C may consist of (that is, contain only) components A, B, and C; or the article may contain not only components A, B, and C, but also one or more additional components.

As used herein, the term “derived” means obtained from a source, directly or indirectly. For example, cells may be directly derived from an organism by obtaining a tissue or organ from the organism, then disaggregating the tissue or organ to obtain primary cells. Cells may be obtained indirectly from an organism by, for example but not limited to, obtaining an isolate, typically a single cell isolate from a cell line that was obtained from the organism, then expanding the isolate to obtain a cell line comprising a multiplicity of cells, sometimes referred to as a subclone.

The term “lepidopteran insect”, as used herein, refers to any member of a large order (Lepidoptera) of insects comprising the butterflies, moths, and skippers that as adults have four broad or lanceolate wings usually covered with minute overlapping and often brightly colored scales and that as larvae are caterpillars. Exemplary lepidopteran insects include but are not limited to, Spodoptera frugiperda, Bombyx mori, Heliothis subflexa, and Trichoplusia ni.

As used herein, the term “substantially” refers to a variation of no more than plus or minus ten percent relative to the named item or items. For example but not limited to, a cell line that has an average cell diameter that is between 90% and 110% of the average diameter of Sf9 cells, based on a statistically significant sample size, when the cell line and the Sf9 cells are propagated under the same conditions, and the average cell diameter is determined as described herein; or a cell line that has a cell density that is between 90% and 110% of the cell density of Sf9 cells, based on a statistically significant sample size, when the cell line and the Sf9 cells are propagated under the same conditions, and the cell density is determined as described herein.

### DETAILED DESCRIPTION

Among other things, the present disclosure provides high productivity insect cell lines. In some embodiments, the present disclosure provides methods for making, preparing, and/or obtaining high productivity cell. In some embodiments, high productivity insect cell lines, disclosed herein, are used for AAV production and/or protein expression. In some embodiments, high productivity insect cell lines are derived from cells and/or organisms. In some embodiments, high productivity insect cell lines are derived from cells and/or organisms with low productivity for AAV production and/or protein expression.

It is to be understood that both the foregoing general description and the following detailed descriptions are illustrative and exemplary only and are not intended to limit the scope of the disclosed teachings. The section headings used herein are for organizational purposes only and are not to be construed as limiting the subject matter of the disclosed teachings.

In the Summary above, the Detailed Description, the accompanying Figures, and the claims below, reference is made to particular features (including method steps) of the current teachings. It is to be understood that the disclosure in this specification includes possible combinations of such particular features. For example but not limited to, where a particular feature is disclosed in the context of a particular embodiment of the current teachings, or a particular claim, that feature may also be used, to the extent possible, in combination with and/or in the context of other particular embodiments, and in the current teachings in general.

Where reference is made to a method comprising two or more combined steps, the defined steps can be performed in any order or simultaneously (except where the context excludes that possibility), and the method may include one or more additional steps which are carried out before any of the defined steps, between two of the defined steps, or after all of the defined steps (except where the context excludes that possibility).

Cells propagated in vitro can be broadly categorized as either primary cells or continuous cell lines, also referred to as an established cell line. Primary cells may be obtained by isolating an organ or tissue from an organism and disaggregating it to create a mixture of individual cells. When primary cells are propagated in culture, they divide only a limited number of times before losing their ability to proliferate, a genetically determined event known as senescence. Some cells, however, undergo a process called transformation and acquire the ability to divide indefinitely. These cells are referred as transformed cells or continuous cells. Compared to the naturally occurring cells found in the tissue or organ from which they were derived, continuous cell lines typically have genetic abnormalities such as aneuploidy or heteroploidy, and lack contact inhibition and anchorage dependence often seen with primary cells.

Since it was first described in the peer-reviewed literature in the early 1980's, the baculovirus-insect cell system (BICS) has become a widely recognized and heavily utilized recombinant protein production platform. The advantages of the BICS include its flexibility, speed, simplicity, eukaryotic protein processing capabilities, and ability to produce multi-subunit protein complexes. For nearly 30 years, the BICS was used mainly to produce recombinant proteins for basic research in academic and industrial labs. More recently, however, the BICS emerged as a bona fide commercial manufacturing platform, which is now being used to produce several biologics licensed for use in human (CERVARIX®, PROVENGE®, GLYBERA®, FLUBLOK®, LUXTURNA, and ZOLGENSMA) or veterinary (PORCILIS® PESTI, BAYOVAC CSF E2®, CIRCUMVENT® PCV, INGELVAC CIRCOFLEX® and PORCILIS® PCV) medicine. In addition, the BICS is being used to produce several other biologics, including noroviral, parvoviral, Ebola viral, respiratory syncytial viral, and hepatitis E viral vaccine candidates in various stages of human clinical trials.

The insect cell lines most commonly used as hosts in the BICS are derived from the cabbage looper, Trichoplusia ni (Tn), or fall armyworm, Spodoptera frugiperda (Sf), and most biologics manufactured with the BICS are produced using the latter. The original Sf cell line, designated IPLB-SF-21, also known as Sf-21, was derived from pupal ovaries (Vaughn et al. 1977). Other commonly used Sf cell lines include Sf9 (a subclone of IPLB-SF-21), and its daughter subclones, including Super 9 and Sf900+, also known as EXPRESSF+® (U.S. Pat. No. 6,103,526A). The original Tn cell line, designated TN-368, was derived from ovarian tissue isolated from newly emerged virgin female moths (Hink 1970). Other commonly used Tn cell lines include BTI-Tn-5B1-4 (commercialized as HIGH FIVE™) and Tni PRO cells.

Since the first report that Sf9 cells were able to produce AAV vectors after co-infection with three recombinant baculoviruses (rBVs) respectively carrying AAV rep coding sequences, AAV cap coding sequences, and a gene of interest (GOI) flanked by AAV ITRs (Urabe, Ding and Kotin 2002), there have been reports of significant improvements to the technology of AAV production in insect cells. The inventors designed an artificial intron comprising an insect promoter and inserted it into both of the AAV rep and cap genes to stabilize the rep construct and increase the VP1 expression levels, which significantly improved the quality of AAV vectors produced in Sf9 cells (Chen 2008). A MonoBac system was reported that all the components necessary for AAV production in the insect cells were cloned into a single baculovirus and AAV vectors were produced by a single baculovirus infection (Galibert et al. 2021). A OneBac system was reported that the Sf9 cell line stably transfected with the AAV rep-cap genes was able to produce AAV vectors by infection of the cells with a recombinant baculovirus carrying a gene of interest (GOI) flanked by the AAV ITRs (Mietzsch et al. 2014).

All these baculovirus-mediated AAV production systems used Sf9 derived cell lines from different sources. After comparative studies of a variety of insect cell lines including several Sf9 cell lines from different CROs, the HIGH FIVE, and the Tni PRO cell lines, our results indicated that the Sf9 derived cell line from Expression Systems (Davis, CA) was the most productive in AAV production among the cell lines tested. However, there is a limit in passaging the cells to maintain high productivity and quality of the AAV vectors. Generally, low passage number of the cells are used to obtain high productivity and high quality of the AAV vectors. These observations are supported by several previous reports that low-passage cells were smaller in size but expressed up to ˜20-fold more protein in total (Joosten and Shuler 2003). Clemm (In: O'Reilly D R., Miller L K, Luckow V A (eds) Baculovirus expression vectors: a laboratory manual. W. H. Freeman, New York, pp 241-248) stated that high-passage insect cells in serum-free media generally exhibit reduced yields and require higher m.o.i. to achieve the same level of protein production as recently adapted cells. Expression of baculovirus genes appears to down-regulate in high-passage cells, compared with that in low-passage cells.

In some embodiments, the present disclosure provides cell lines. In some embodiments, the present disclosure provides genetically modified cell lines. In some embodiments, a genetically modified cell line is characterized by an increased level of AAV vector productivity and/or protein expression levels (e.g., relative to an unmodified cell line or a parental cell). In some embodiments, a genetically modified cell line is characterized by an increased level of AAV vector productivity and/or protein expression levels as compared to a parental cell line. In some embodiments, a genetically modified cell line described herein is particularly useful for production of AAV vectors, vaccines, recombinant proteins and/or biologics for human and/or veterinary use. In some embodiment, a genetically modified cell line and/or parental cell line is derived from a cell. In some embodiments, a cell comprises an insect cell. In some embodiments, a genetically modified cell line and/or parental cell line is derived from an organism. In some embodiments, an organism is an insect.

### Baculovirus Immediate-Early Gene, IE1

Autographa californica multiple nucleopolyhedrovirus (AcMNPV) is a type species of the genus Alphabaculovirus. AcMNPV expresses two immediate-early genes during the early phase of infection, a nonspliced 1.9-kb transcript and a spliced 2.1-kb transcript, which encode IE1 and IE0 proteins respectively. These two gene products differ as IE0 contains an additional 54 amino acids at the amino terminus. In addition, IE1 is expressed during early and late infection, whereas IE0 is expressed early in infection. Furthermore, IE0 transactivates a 39K promoter in the presence of cis-linked homologous region (hr) 5 enhancer, while IE1 transactivates a delayed early 39K gene in the presence and absence of the homologous region (hr) enhancers.

Sebastian et al. described that a recombinant baculovirus comprising a strong IE1 expression cassette and a foreign gene expression cassette comprising hr1 enhancer and a composite promoter (p6.9-p10 or pB2(9)p10) was able to increase the expression level of the foreign gene after infection of insect cells (See, e.g., U.S. Pat. No. 9,982,239). However, Shultz et al. reported that IE1 was required for baculovirus early replication events that triggered apoptosis in permissive and nonpermissive insect cells, indicating that IE1 expression may have a negative impact on insect cell survival (See, e.g., Gomez-Sebastian, Lopez-Vidal and Escribano 2014). IE1 sequence is known in the art (see, e.g., Chisholm et al., J. Virol. 62:3193-3200 (1988); Carson et al., J. Virol. 65:945-951 (1991); Rodems et al., J. Virol. 71:9270-9277 (1997); Olson et al., J. Virol. 77:5668-5677 (2003); U.S. Pat. No. 9,982,239). In some embodiments, an IE1 sequence is or comprises the amino acid sequence of GenBank Accession No. P11138.3 or sequence described by Chisholm et al., J. Virol. 62:3193-3200 (1988); Carson et al., J. Virol. 65:945-951 (1991); Rodems et al., J. Virol. 71:9270-9277 (1997); Olson et al., J. Virol. 77:5668-5677 (2003), U.S. Pat. No. 9,982,239, or a portion thereof (e.g., a fragment at least 90%, 92%, 94%, 96%, 98%, or 99% length of a disclosed sequence). In some embodiments, an IE1 sequence is or comprises an amino acid sequence at least 90%, 91%, 92%, 93%, 94%, 95%, 96%, 97%, 98%, or 99% identical to the sequence of GenBank Accession No. P11138.3, or sequence described by Chisholm et al., J. Virol. 62:3193-3200 (1988); Carson et al., J. Virol. 65:945-951 (1991); Rodems et al., J. Virol. 71:9270-9277 (1997); Olson et al., J. Virol. 77:5668-5677 (2003), U.S. Pat. No. 9,982,239, or a portion thereof (e.g., a fragment at least 90%, 92%, 94%, 96%, 98%, or 99% length of a disclosed sequence). In some embodiments, an IE1 sequence is or comprises a nucleotide sequence encoding the amino acid sequence of GenBank Accession No. P11138.3 or sequence described by Chisholm et al., J. Virol. 62:3193-3200 (1988); Carson et al., J. Virol. 65:945-951 (1991); Rodems et al., J. Virol. 71:9270-9277 (1997); Olson et al., J. Virol. 77:5668-5677 (2003), U.S. Pat. No. 9,982,239, or a portion thereof (e.g., a fragment at least 90%, 92%, 94%, 96%, 98%, or 99% length of a disclosed sequence). In some embodiments, an IE1 sequence is or comprises a nucleotide sequence encoding an amino acid sequence at least 90%, 91%, 92%, 93%, 94%, 95%, 96%, 97%, 98%, or 99% identical to the sequence of GenBank Accession No. P11138.3, or sequence described by Chisholm et al., J. Virol. 62:3193-3200 (1988); Carson et al., J. Virol. 65:945-951 (1991); Rodems et al., J. Virol. 71:9270-9277 (1997); Olson et al., J. Virol. 77:5668-5677 (2003), U.S. Pat. No. 9,982,239, or a portion thereof (e.g., a fragment at least 90%, 92%, 94%, 96%, 98%, or 99% length of a disclosed sequence).

In some embodiments, the present disclosure provides a cell line (e.g., an insect cell line) expressing IE1 (e.g., constitutively expressing IE1). In some embodiments, a cell line is derived from an insect. In some embodiments, a cell line is derived from a lepidopteran insect. In some embodiments, a lepidopteran insect comprises Spodoptera frugiperda, Trichoplusia ni, or Bombyx mori. In some embodiments, a cell line is derived from Spodoptera frugiperda Sf-9 designated ATCC CRL-1711. In some embodiments, the present disclosure provides a cell line comprising a baculovirus IE1 coding sequence. In some embodiments, a genetically modified cell line comprises a baculovirus IE1 coding sequence. In some embodiment, an insect cell line comprises a baculovirus IE1 coding sequence. In some embodiments, a genetically modified insect cell line comprises a baculovirus IE1 coding sequence.

### Characterization of Modified Cell Line

In certain embodiments, a genetically modified cell line described herein is characterized by having substantially different cell density and/or doubling time as parental cells from which the cell line was derived. In some embodiments, a genetically modified cell line is characterized by having substantially different cell density and/or doubling time as parental cells from which the cell line was derived when: (1) both cell lines are propagated under the same conditions, (2) the comparison is performed as described herein, and/or (3) the comparisons are based on a statistically significant sample size. In certain embodiments, a genetically modified cell line described herein is characterized by higher level of production of AAV vectors and/or higher level of expressed recombinant proteins than the parental cells from which the cell line was derived, e.g., when each are infected with same recombinant baculovirus (rBVs) under the same conditions and the comparison is performed according to the present Examples.

In some embodiments, a cell line described herein is characterized by higher AAV production yields than a parental insect cell line. In some embodiments, a cell line described herein is characterized by higher AAV protein expression levels than a parental insect cell line.

### Methods of Obtaining Modified Cells

According to some embodiments, exemplary methods for obtaining modified cells include culturing modified cells in a media comprising a selection agent and isolating modified cells from unmodified cells. In some embodiments, a selection agent is or includes an antibiotic agent.

In some embodiments, cells are cultured with an appropriate cell culture media that includes an antibiotic agent (e.g., G418). Exemplary antibiotics compounds include neomycin, zeocine, hygromycin, blasticidin, puromycin, phleomycin, and G418 (geneticin).

In some embodiments, a first culture composition is incubated under conditions suitable for the cells to grow and divide; and for a sufficient period of time to allow an antibiotic agent (e.g., G418) to kill any cells that does not include a resistant gene, e.g., a NeoR_KanR gene.

In some embodiments, methods described herein include, aliquoting cells or culture media from a culture, and testing for a presence of a gene. In some embodiments, methods described herein includes, aliquoting cells or culture media from a culture, and testing for a presence of an IE1 gene. In some embodiments, methods to test for a presence of a gene are known in the art, e.g., quantitative PCR, nested PCR, or PCR, followed by analysis of resulting amplicons for the presence or absence of IE1 coding sequence specific amplification products, and SDS-PAGE followed by Western blot to detect the IE1 protein.

In some embodiments, methods include combining cells containing a IE1 gene with culture media that does not contain an antibiotic agent (e.g., G418) to form a second culture composition. In some embodiments, methods further include, incubating a second culture composition under conditions to allow cells to grow and divide. In some embodiments, cells or culture media from a second culture composition are tested for a presence or absence of a IE1 gene and encoded protein.

In some embodiments, methods further include, expanding cultured cells to obtain a cell line that contains the IE1 gene.

In certain embodiments, methods for obtaining a genetically modified cell line including a IE1 gene comprises: (i) transfecting a population of lepidopteran insect cells (e.g., Spodoptera frugiperda or Trichoplusia ni cells) with a plasmid V432-pcDNA-IE11-pIE1-NeoR_KanR; (ii) killing non-transfected cells with an antibiotic agent (e.g., G418) by culturing cells with culture media including an antibiotic agent (e.g., G418) to form a first culture composition; (iii) incubating a first culture composition under conditions suitable for the cell to grow and divide, thereby generating a plurality of cells; (iv) removing a portion of cells or cell culture media and testing for the presence of IE1 gene; (v) combining at least a portion of the plurality of cells from a first culture composition with cell culture media without an antibiotic agent (e.g., G418) to form a second culture composition; and (vi) incubating a second culture composition under conditions suitable for a cells to grow and divide, thereby obtaining a cell line characterized by the presence of the IE1 gene.

In certain embodiments, methods for obtaining a genetically modified cell line including a IE1 gene comprises: (i) transfecting a population of lepidopteran insect cells (e.g., Spodoptera frugiperda or Trichoplusia ni cells) with a plasmid V432-pcDNA-IE11-pIE1-NeoR_KanR; (ii) killing non-transfected cells with an antibiotic agent (e.g., G418) by culturing cells with culture media including an antibiotic agent (e.g., G418) to form a first culture composition; (iii) incubating a first culture composition under conditions suitable for the cell to grow and divide, thereby generating a plurality of cells; (iv) combining at least a portion of the plurality of cells from a first culture composition with cell culture media without an antibiotic agent (e.g., G418) to form a second culture composition; and (v) incubating a second culture composition under conditions suitable for a cells to grow and divide, thereby obtaining a cell line characterized by the presence of the IE1 gene.

In some embodiments, an individual cell or small groups of cells, e.g., 2 cells, 3 cells, 4 cells, 5 cells, 10 or fewer cells, or 20 or fewer cells (including every whole number between 1 and 20) are isolated from a colony of cells that is resistant to an antibiotic agent (e.g., G418). Non-limiting examples of techniques for isolating a single cell or small numbers of cells include: limiting dilution cloning (sometimes referred to as cloning by serial dilution), cloning cells in soft agar and subsequently picking cell colonies, cell sorting to isolate single or small numbers of cells, laser capture microdissection (LCM), using micropipettes (for example but not limited to ultra-thin capillaries) to manually capture individual or small numbers of cells, microfluidics, or using micromanipulators to microscopically assist the selection of single or small numbers of cells. In some embodiments, isolating a single cell comprises limited dilution cloning.

Those in the art will appreciate that conditions suitable for growing a particular cell type are readily ascertainable from a variety of sources, for example but not limited to, cell culture manuals, commercial cell banks, or vendors of culture media and/or plastic ware. Appropriate cell culture conditions may also easily be determined using methods known in the art.

All publications, patent applications, patents, and other references mentioned herein, including GenBank sequences, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1. Construction of Plasmid Containing the Baculovirus IE1 Coding Sequence

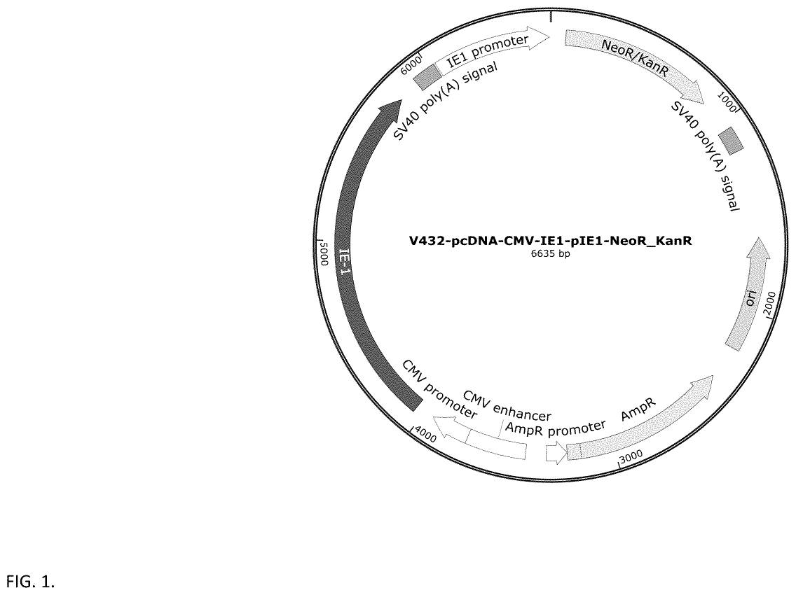

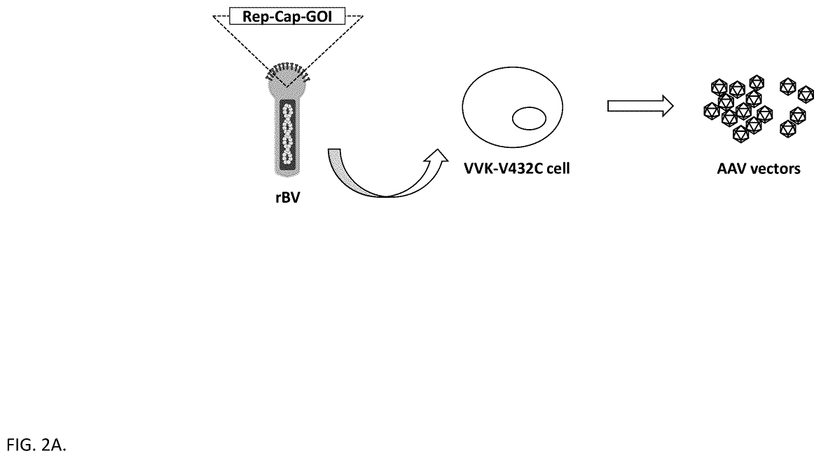

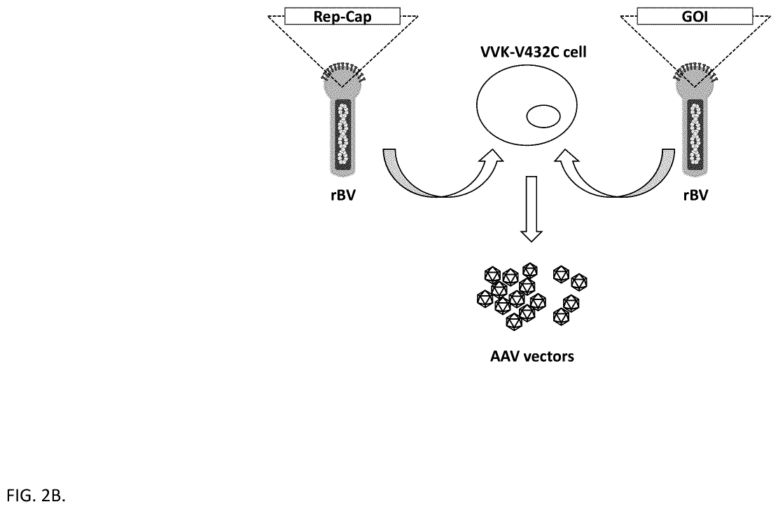

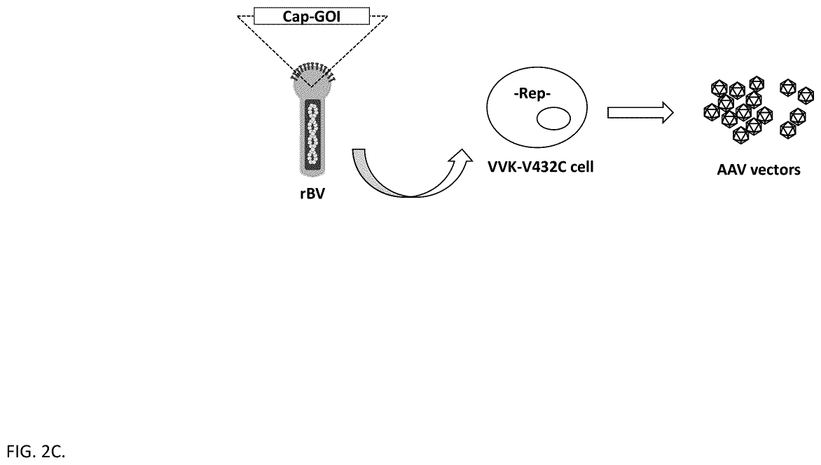

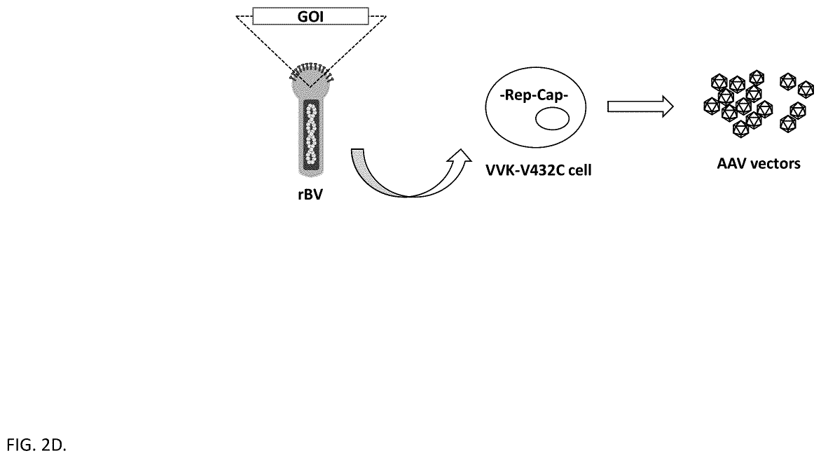

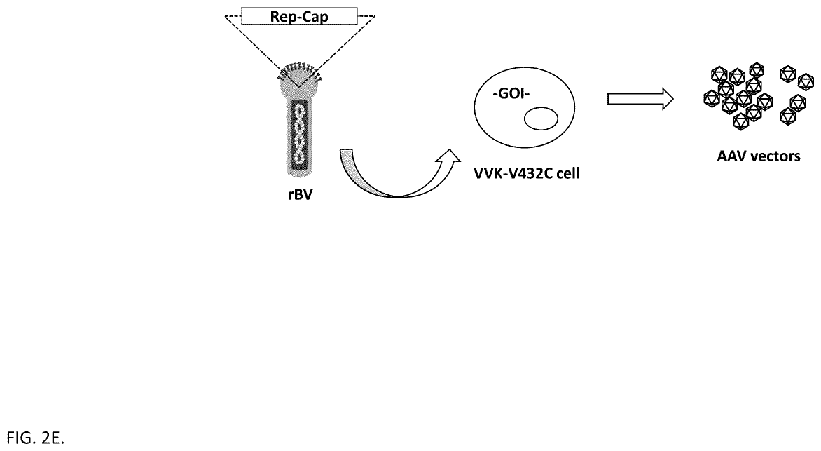

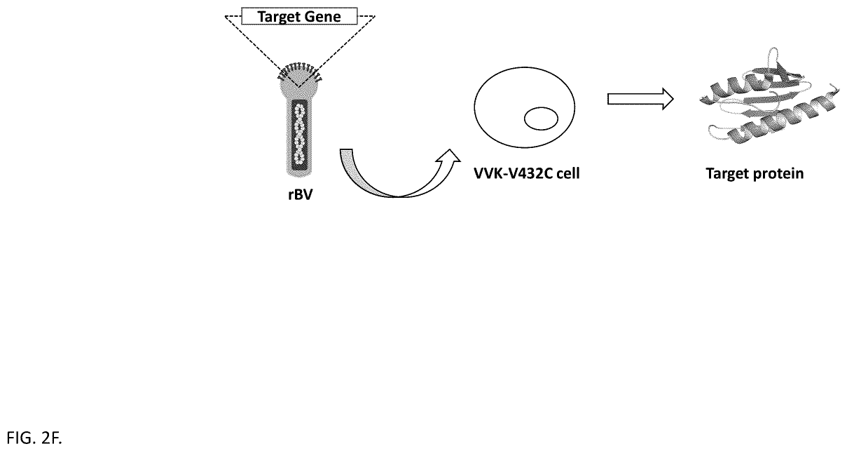

Plasmid V422 that contains a SV40 polyadenylated sequence, pIE1 promoter (SV40 pA-pIE1), and resistant gene NeoR/KanR for G418 selection was used as starting material. The plasmid was digested with AvrII and BglII to isolate the backbone fragment containing the NeoR/KanR genes. A PCR fragment containing the CMV promoter was amplified with forward primer #5421 (5′-GTCGACGGATCGGGAGATCTTATTAATAGTAATCAATTAC-3′ (SEQ ID NO: 1)) and reverse primer #5422 (5′-TTGCGTCATGGTGGCGGTTGGAGGCTGGATCGGTC-3′ (SEQ ID NO: 2)) and plasmid pFB-CMV-GFP as template. A second PCR fragment containing the baculovirus IE1 coding sequence was amplified with forward primer #5423 (5′-CAACCGCCACCATGACGCAAATTAATTTTAACGCG-3′ (SEQ ID NO: 3)) and reverse primer #5445 (5′-TATGATCCTCGACTTAATTAAATTCGAATTTTTTATATTTACAATTTAGTTTTTGTTCC G-3′ (SEQ ID NO: 4)) and a bacmid DNA as template. A third PCR fragment containing the SV40 pA-pIE1 fragment was amplified with forward primer #5425 (5′-ATTAAGTCGAGGATCATAATCAGCCATACCACATT-3′ (SEQ ID NO: 5)) and reverse primer #5281 (5′-GCTTTTTGCAAAAGCCTAGGCTTGGTTGTTCACGATCTTG-3′ (SEQ ID NO: 6)) and plasmid V422 as template. These three PCR fragments were purified and joined together through a PCR reaction with primers #5421 and #5281. The joined PCR fragment was purified and cloned into the V422 backbone through HiFi assembly reaction to create V432-pcDNA-CMV-IE1-pIE1-NeoR_KanR. This plasmid was used for stable transfection into ATCC Sf9 cell line CRL-1711 (FIG. 1).

### Example 2. Adaptation of ATCC Sf9 Cell Line into Serum-Free Suspension Culture

One vial of the cell line purchased from ATCC (CRL-1711, Lot 63655513) was first rinsed with 10 mL of Grace Insect media containing 10% FBS and then resuspended in 6 mL of the same media and transferred to 2 wells of a 6-well plate (1/8/2018). The cells were cultured at 28° C. for 4 days.

6 mL old media containing viable floating cells from a 6-well plate were harvested and transferred into a 150-mL size Corning storage bottle followed by adding 4 mL of ESF 921 media without FBS. Leftover adherent cells in a 6-well plate were maintained with Grace Insect media containing 10% FBS at 28° C. Suspension cell culture conditions were set at 28° C., 140 rpm in serum-free ESF921 media.

After culturing for 3 additional days, the cells were added with another 5 mL of ESF 921 media. At day 11, the cell density reached 4×10⁵ cells/mL with 72% viability. At day 13, additional 2 mL floating cells were collected from the adherent culture in the 6-well plate and added to the suspension culture followed by 5 mL of ESF921 media. The suspension cells were cultured until day 15. Because the cell density and viability were not improved, the whole cell culture of 25 mL were spun down to remove 20 mL old media. The cells were resuspended gently in the leftover 5 mL old media and added with 5 mL fresh ESF 921 media to obtain a density of 1×10⁶ cells/mL.

The suspension cells were cultured until day 18, when the cell density reached 2.08×10⁶ cells/mL and viability increased to 82%. The cells were added with 10 mL fresh ESF921 media and kept culturing until day 21. Another 10 mL fresh ESF921 media were added. At day 23, the cell culture was expanded to 40 mL after adding another 10 mL of ESF921 media.

At day 25, the 40 mL suspension culture reached 7×10⁵ cells/mL and viability at 92%. In order to increase the cell density for better cell growth, the culture was spun down to remove all the old media and resuspended in 20 mL of fresh ESF921 media to obtain 1.4×10⁶ cells/mL. At day 28, the suspension culture reached 2.37×10⁶ cell/mL and viability at 93% and 5 mL fresh ESF921 media was added to provide nutrients. At day 30, another 5 mL ESF921 media were added. At day 32, 30 mL suspension culture reached 3.00×10⁶ cells/mL and viability at 96%.

The culture was diluted with 30 mL fresh ESF921 media to obtain 1.5×10⁶ cells/mL. At day 35, the 60 mL cell culture reached 2.0⁶×10⁶ cells/mL and viability at 86% and was added with 10 mL fresh ESF921 media to provide nutrients. At day 37, the cell culture reached 2.3×10⁶ cells/mL and viability at 83%. Because the cells were growing slow, half of the 70 mL old media were removed by gentle centrifugation and replaced with half of fresh ESF921 media to provide more nutrients. At day 39, the cell density reached 3.04×10⁶ cells/mL and viability at 86%. The 70 mL culture was transferred to a fresh culture bottle and added with 35 mL fresh ESF921 media to adjust cell density to 2×10⁶ cells/mL.

At day 41, the 105 mL culture reached 2.90×10⁶ cells/mL but cell viability decreased to 68%. 50 mL of the old media were removed by gentle centrifugation and replaced with 50 mL fresh ESF921 media. At day 43, the cell density reached 3.43×10⁶ cells/mL but viability decreased to 57%. The cells were added with 50 mL fresh ESF921 media and kept at 28° C., 140 rpm.

The next day, the cell viability decreased to 46%. The whole culture was centrifuged at 1,000 rpm to remove 2/3 of the old media and the cells were resuspended in 50 mL old media followed by 50 mL fresh ESF921 media. At day 45, the 100 mL culture reached 3.3×10⁶ cells/mL and the viability increased slightly to 56%. Another 50 mL old media were removed and replaced with 50 mL fresh ESF921 media. At day 46, the cell density increased to 3.6×10⁶ cells/mL and the viability to 67%. The cells were centrifuged to remove 70 mL old media and resuspended in 30 mL old media. The cells were transferred to a 1-liter size culture bottle and added with 170 mL fresh ESF921 media to obtain a total volume of 200 mL. At day 47, the cell density remained at 1.55×10⁶ cells/mL but the cell viability increased to 71%. The culture was centrifuged to remove 50 mL old media and replaced with 50 mL fresh ESF921 media.

At day 48, the cell density was at 1.40×10⁶ cells/mL and the viability was at 73%. In order to increase the cell density for better cell growth, the cells were centrifuged to remove 100 mL old media and added with 50 mL fresh ESF921 media to obtain 150 mL culture volume. At day 49, the cell density reached 2.84×10⁶ cells/mL and the viability increased to 80%. The culture was centrifuged to remove 100 mL old media and replaced with 100 mL fresh ESF921 media in a fresh 1-liter size culture bottle and kept at 28° C., 140 rpm. At day 50, another 50 mL fresh ESF921 media was added to obtain a total volume of 200 mL. At day 51, the cell density was at 1.45×10⁶ cells/mL and viability at 82%. 100 mL old media were removed and replaced with 50 mL fresh ESF921 media to obtain a culture volume of 150 mL. At day 52, the cell density increased to 2.33×10⁶ cells/mL and cell viability increased to 90%. The cells were centrifuged to remove 50 mL old media and replaced with 50 mL fresh media in a fresh 1-liter size culture bottle. The cells were cultured at 28° C., 140 rpm.

At day 53, the cell density reached 2.79×10⁶ cells/mL and viability increased to 87%. 100 mL old media were removed by centrifugation and replaced with 100 mL fresh ESF921 media and cells were kept culturing. At day 54, the cell density remained similar but viability increased to 93%, indicating good adaption to the suspension serum-free media. 100 mL old media were removed and replaced with 100 mL fresh media. At day 55, the cell density increased to 3.34×10⁶ cells/mL and viability at 90%. 100 ml old media were removed and replaced with 100 mL fresh media. At day 56, cell density increased to 4×10⁶ cells/mL and viability remained at 91%, indicating a good adaptation in the serum-free suspension culture conditions. 50 mL cells were centrifuged gently to remove the old media and then resuspended in 10 mL freeze media (4.5 mL old media+4.5 mL fresh media+1 mL DMSO).

The resuspended cells were aliquoted into 10 vials and frozen down at −80° C. overnight and transferred to liquid nitrogen for long term storage. For the remaining 100 mL cells, 75 mL old media were removed by gentle centrifugation and replaced with 75 mL fresh media. The cells were cultured further. At day 57, the cell density reached 6.7×10⁶ cells/mL and viability at 97%. The cells were diluted with 100 mL fresh media to obtain a total volume of 200 mL. At day 58, the cell density was at 5.4×10⁶ cells/mL and viability remained at 97%. 100 mL cells were centrifuged and resuspended into 20 mL freeze media. The resuspended cells were aliquoted into 20 vials and kept in liquid nitrogen for long term storage.

After nearly 2 months of adaptation, the ATCC CRL-1711 Sf9 cells were fully adapted to the serum-free ESF921 media in suspension culture with 97% viability and reached density of 7×10⁶ cells/mL with about 28 hour doubling time.

### Example 3. Stable Transfection of Adapted ATCC Sf9 Cells with Plasmid V432-pcDNA-CMV-IE1-pIE1-NeoR_KanR

In these experiments, fully serum-free adapted suspension Sf9 cells were used for the stable transfection. The cells were cultured to 5×10⁶ cells/mL with 96% viability and then were diluted to 1×10⁶ cells/mL in ESF921 media. The diluted cells were added to a 6 well-plate at 2 mL/well. The Sf9 cell line from Expression Systems was used as control in a second 6-well plate. To perform the transfection, 22.5 μg plasmid DNA and 22.5 μL transfection reagent GenJet (SignaGen Laboratories) were each diluted in 900 μL ESF921 media and then mixed together.

After incubation at room temperature for 20 min, 7.2 mL of fresh ESF921 media were added to the DNA-GenJet mixture. After pipetting up and down several times, old media from the 6-well plates were removed and 1 mL/well of the DNA-GenJet mixture was added to each well. After incubation at 28° C. overnight, 1 mL/well of fresh ESF921 media was added. The cells were cultured for a total of 3 days and the cells from each well were dislodged by pipetting and expand to one 10-cm cell culture plate with 15 mL ESF921 media. Selection drug G418 at concentration of 800 μg/mL, 600 μg/mL, and 400 μg/mL were each added to two 10-cm plates, one plate with 5% FBS and the other without FBS.

Every 4 to 5 days old media were removed and replaced with fresh ESF921 media containing G418 at the indicated concentration with or without 5% FBS. After 18 days of G418 selection, cell colonies were formed in plates with G418 at 400 μg/mL and 200 μg/mL with ESF921 media containing 5% FBS. Cell colonies were picked using a 20-μL pipet tip to scratch the cells at the same time suck the cells into the tip. The collected cells were transferred to a 96-well plate, one colony/well in 200 μL ESF921 media with 5% FBS. A total of 30 clones were picked. Twelve clones were expanded to 24-well plates with 1 mL/well ESF921 media plus 5% FBS. Among them 3 clones were expanded to 6-well plate and them to 10-cm plates. Once the cells were about 50% confluent, they were dislodged and transferred to suspension culture in 150-mL bottle with 10 mL volume. Two clones were grown well in suspension culture. Once the cell density reached 2×10⁶ cells/mL, 1 mL cells of each clone were collected for genomic DNA extraction and PCR amplification to identify the IE1 integration. The remaining cells were expanded and then frozen in liquid nitrogen for long term storage.

### Example 4. Verification of IE1 Coding Sequence in Purified Genomic DNA of the Stable Clones

Genomic DNA was purified from the stable clones with the QIAGEN—Blood & Cell Culture DNA Midi Kit according to the manufacturer's protocol. Briefly, two mL of each suspension cell culture with cell density of about 8×10⁶ cells/mL were collected and mixed respectively each with 2 mL ice-cold Buffer C1 and 6 mL ice-cold distilled water in a 15 mL tube by inverting the tube 5 times and incubate on ice for 10 min. The nuclear pellets were collected by centrifuge the lysate at room temperature for 10 min at 2700 rpm (1300×g) in a tabletop Beckman centrifuge and the supernatants were discarded. To each nuclear pellet, another 1 ml of ice-cold buffer C1 and 3 ml of ice-cold distilled water were added to resuspend the pelleted nuclei by vortexing and centrifuged again at room temperature for 10 min at 2700 rpm (1300×g) to remove the supernatant. Then 1 ml buffer G2 was added to resuspend each nuclei by pipetting up-down with P-1000 pipet until the nuclei were fully dissolved.

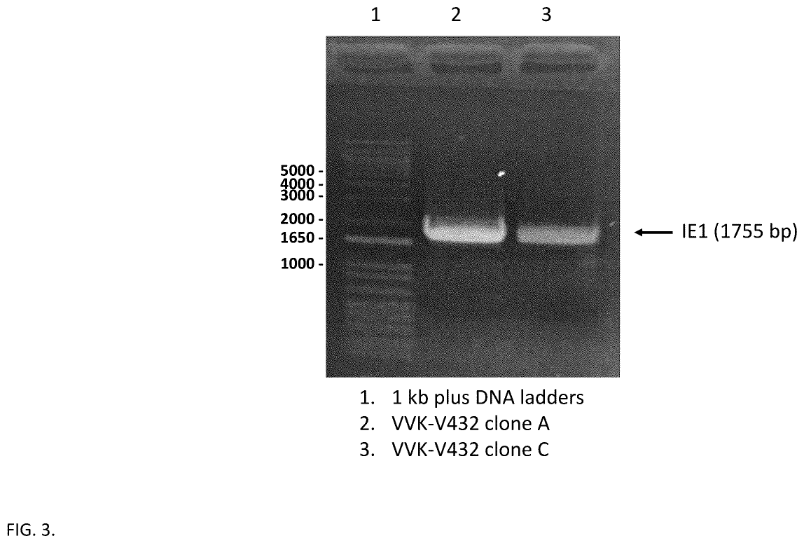

Another 4 mL of Buffer G2 was added to each tube of the nuclei followed by 95 μl proteinase K, mixed and incubated at 50° C. for 60 min. to digest the proteins. The Midi columns were each equilibrated with 4 ml of Buffer QBT and the digested samples were loaded respectively on the columns. After washing twice with 7.5 mL of Buffer QC, the genomic DNA was eluted with 5 mL pre-warmed at 50° C. buffer QF and precipitated by adding 3.5 ml of isopropanol. The DNA pellets were collected by centrifugation at 8,000 rpm for 15 min. at 4° C. and supernatants discarded. The DNA pellets were rinsed each with 4 ml of 70% ethanol and each pellet was dissolved in 1 ml TE buffer. The IE1 coding sequence (1775 bp) was PCR amplified from the purified genomic DNA with forward primer #5423 (5′-CAACCGCCACCATGACGCAAATTAATTTTAACGCG-3′ (SEQ ID NO: 3)) and reverse primer #5424 (5′-ATTATGATCCTCGACTTAATTAAATTCGAATTTTT-3′ (SEQ ID NO: 7)). The results are shown in FIG. 3.

### Example 5. Continuously Passaging the Genetically Modified Cells to Shorten the Cell-Doubling Time

The isolated cell clones VVK-V432A and VVK-V432C exhibited a doubling time of about 28 hours. In order to shorten the cell doubling time, a continuously passaging method was employed to shorten the cell-doubling time. After verification of the IE1 presence in the cells, initial cells cultured in media without antibiotic G418 were designated as passage 1. VVK-V432 clone C was used for the continuous passaging.

The cells were cultured to 2 to 4×10⁶ cells/mL and diluted 1:1 daily with fresh culture media in the week days. The cell number was monitored daily in the week days. During weekend, the cells were diluted 1:4 with culture media. After a total of 88 passages, the cell doubling time reached at about 24 hours. Cryopreservation of the cells was performed and the cells were stored at liquid nitrogen for long term storage.

### Example 6. Generation of Recombinant Baculoviruses Carrying AAV Rep and Cap Gene and GOI Respectively in the VVK-V432C Cell Line

In these experiments, recombinant baculoviruses (rBVs) were generated according to manufacturer's protocols (Invitrogen). Briefly, the plasmids were respectively diluted into 1 ng/μl in TE buffer and 2 μl of each diluted plasmid was transformed into DH10Bac competent bacteria. After 48-hour incubation, white colonies were picked and miniprep Bacmid DNAs were prepared. The miniprep Bacmid DNAs were respectively transfected into the VVK-V432C cells for 4 days. Supernatants from all the transfected cells were harvested as rBV stocks. The rBV stocks were amplified once and titers were determined with quantitative PCR (QPCR) method using primers corresponding to the gentamicin gene. Briefly, 50 μL rBV was mixed with 50 μL 0.2% SDS and incubated at 95° C. for 30 min to break the viral particles and release viral DNA molecules. After cool down to room temperature, the baculoviral samples were diluted with QPCR dilution buffer (10 ug/mL yeast tRNA, 0.01% Tween 80, 10 mM Tris-HCl, pH8.0, 1 mM EDTA) and assayed with QPCR method with forward primer #1727 (5′-AAACCTGGGCAGAACGTAAG-3′ (SEQ ID NO: 8)) and reverse primer #1728 (5′-TAAGACATTCATCGCGCTTG-3′ (SEQ ID NO: 9)).

The QPCR titers (genome copies/ml) were converted to plaque forming units (pfu/ml) by dividing with a factor of 20 (empirically determined). The results in Table 1 demonstrate that recombinant baculoviruses were generated to high titers in the genetically modified VVK-V432C cells and the titers were comparable to control Sf9 cells.

TABLE 1
Comparison of rBV titers generated in VVK-V432C and control Sf9 cells.

| Item | Cell line | Lot number | rBV name | Titer (pfu/mL) |
|---|---|---|---|---|
| 1 | VVK-V432C | 09132019ICM04 | rBV-GOI-P1 | 1.00E+09 |
| 2 | VVK-V432C | 04102019V218 | rBV-inCap8-inRep-1-P3 | 6.00E+08 |
| 3 | VVK-V432C | 04102019V217 | rBV-CMV-Cre-P1 | 1.00E+09 |
| 4 | VVK-V432C | 06282019AVA13 | rBV-CMV-Luciferase-P1 | 5.40E+08 |
| 5 | VVK-V432C | 04122019V104 | rBV-inCap2-inRep-3-P3 | 1.00E+09 |
| 6 | Control Sf9 | 06282019V290WT | rBV-inCap6-inRep-P1 | 9.20E+08 |
| 7 | Control Sf9 | 06282019V290CATH | rBV-inCap6-inRep-P1 | 8.50E+08 |
| 8 | Control Sf9 | 09132019V108 | rBV-inCap5.2-inRep-6-P3 | 5.60E+08 |

### Example 7. Production of AAV Vectors in VVK-V432C Cells

In these experiments, genetically modified VVK-V432C and the control Sf9 cells were both grown to about 1E+7 cells/ml in ESFAF media (Expression Systems, CA) supplemented with 100 units/ml of penicillin and 100 μg/ml of streptomycin. The cells were then diluted 1:1 with fresh media and infected with 10 moi of rBV carrying Rep-Cap (serotypes 2, 6, or 8) and 5 moi of rBV carrying CMV-Cre for 3 days. The cell pellets were harvested by centrifugation at 2000 rpm for 10 min. and lysed in SF9 lysis buffer (50 mM Tris-HCl, pH 7.4, 2 mM MgCl2, 1% saykosyl, 1% triton X-100, 125 units/ml of benzonase). Cellular DNA was digested by incubating at 37° C. for 60 min.

Cell lysates were cleared by centrifugation at 8000 rpm for 30 min. and loaded onto SW28 centrifuge tubes containing 5 ml of 1.55 g/cc, and 10 ml of 1.32 g/cc of CsCl solutions. After centrifugation at 28,000 rpm for about 20 hours at 15° C., the rAAV-containing fraction was collected by puncturing the centrifuge tube using a syringe needle and subjected to a second round of CsCl ultracentrifugation at 65,000 rpm for about 20 hours. The rAAV-containing fraction was collected again by puncturing the centrifuge tube using a syringe needle and buffer-exchanged with PBS buffer containing 0.001% pluronic F-68 using 2 PD-10 desalting columns to remove the salts and detergents. Vector titers were determined by QPCR assay according to manufacturer's protocol (Applied Biosystems, Foster City, CA). The results presented in Table 2 show that higher titers of rAAV vectors were produced in genetically modified VVK-V432C cell line than the control Sf9 cell line.

TABLE 2
Comparison of AAV vector titers produced
in VVK-V432C and control Sf9 cells.

| | | | | AAV yield |
|---|---|---|---|---|
| Item | Cell line | Sample | AAV name | (vg/L) |
| 1 | VVK-V432C | Crude lysate | AAV2-CMV-Cre | 1.01E+15 |
| 2 | Control Sf9 | Crude lysate | AAV2-CMV-Cre | 9.83E+14 |
| 3 | VVK-V432C | Crude lysate | AAV6-CMV-Cre | 1.89E+15 |
| 4 | Control Sf9 | Crude lysate | AAV6-CMV-Cre | 5.83E+14 |
| 5 | VVK-V432C | Crude lysate | AAV8-CMV-Cre | 3.15E+15 |
| 6 | Control Sf9 | Crude lysate | AAV8-CMV-Cre | 1.19E+15 |
| 7 | VVK-V432C | Purified | AAV2-CMV-Cre | 1.08E+15 |
| 8 | Control Sf9 | Purified | AAV2-CMV-Cre | 5.16E+14 |
| 9 | VVK-V432C | Purified | AAV6-CMV-Cre | 8.46E+14 |
| 10 | Control Sf9 | Purified | AAV6-CMV-Cre | 4.07E+14 |
| 11 | VVK-V432C | Purified | AAV8-CMV-Cre | 2.22E+15 |
| 12 | Control Sf9 | Purified | AAV8-CMV-Cre | 6.40E+14 |

### Example 8. SDS-PAGE Characterization of Purified AAV Vectors Produced in the VVK-V432C Cell Line

In these experiments, VVK-V432C cell produced AAV vectors were compared with control Sf9 cell for the VP1, VP2, and VP3 ratio and product purity in SDS-PAGE electrophoresis. Briefly, purified AAV vectors were mixed with SDS-loading buffer and heated at 95° C. for 5 min. After cooling down to room temperature, the samples were vortexed and centrifuged briefly to collect all liquid to the bottom of the tubes. The AAV samples were loaded 1E+11 vg/lane onto the SDS-gel and subjected to electrophoresis at 100V until the loading dye reached the bottom of the gel. The gels were stained and de-stained with the SimplyBlue SafeStain kit according to Manufacturer's protocol (Invitrogen).

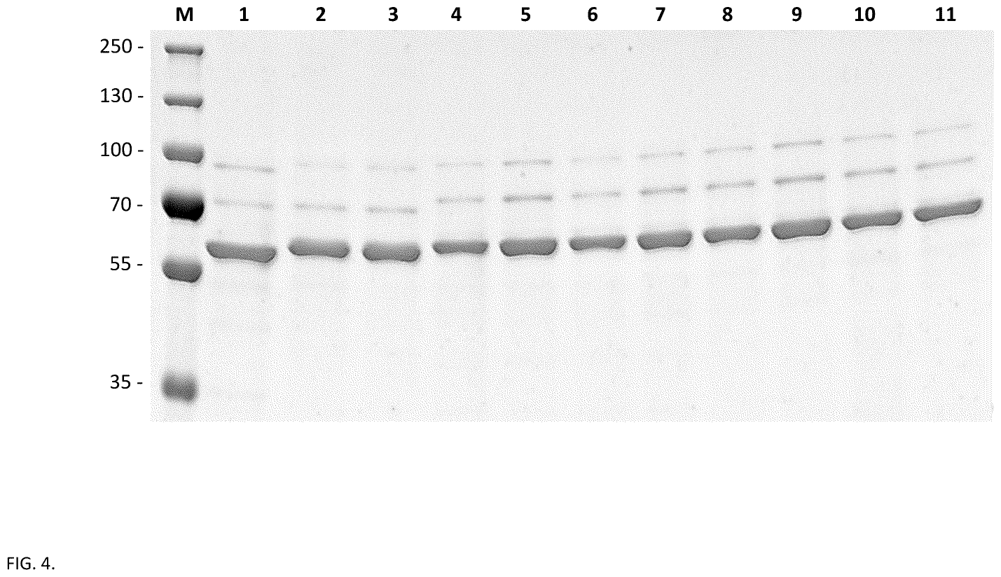

The results in FIG. 4 demonstrate that VVK-V432C cell line produced AAV vectors had correct VP1, VP2, and VP3 ratio as compared with other Sf9 cell lines. There was no difference in product purities, indicating that no extra contaminants were present in the AAV vectors produced with the genetically modified cell line.

### Example 9. Transduction of Mammalian Cells with AAV Vectors Purified from VVK-V432C Cell Line

In these experiments, the infectivity of AAV vectors purified from the genetically modified VVK-V432C, the control Sf9 cell line, and the parental ATCC cell line was compared through transduction assays. Briefly, HEK293 cells were seeded in 24-well plates at 1.5E+5 cells/well in 500 μL volume DMEM media containing 10% FBS and cultured overnight at 37° C. in a CO₂ incubator. The next morning, a 10-fold serial dilution of AAV vectors from 2E+10 vg/mL to 2E+7 vg/mL in serum-free DMEM media containing 20 M etoposide was prepared. After removing the old media from the cells and gently rinsing with 0.5 mL/well serum-free media, the cells were treated with 500 μL/well diluted AAV vectors and cultured overnight in the incubator. The next morning, 500 μl/well of culture media containing 20% FBS were added and the cells were cultured for additional 3 days. The old media were removed from each well and 500 μL of PBS was carefully added to rinse the cells and removed again. Then the cells in each well were resuspend in 500 μL PBS and 100 μL of the resuspended cells from each well were transferred to the 96-well plate.

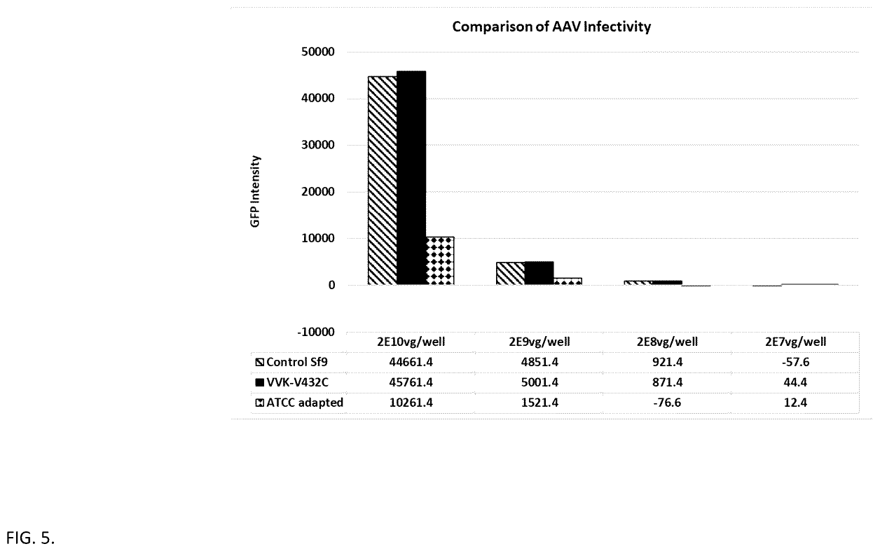

GFP expression was assessed using the TECAN Ultra 384 with fluorescence filter and the GFP intensity was recorded. HEK293 cells without AAV transduction were used as control. The fluorescent readout from all wells were subtracted by the control. As shown in FIG. 5, the results demonstrate that AAV vectors produced in the genetically modified cell line VVK-V432C had essentially the same infectivity as the AAV vectors produced in the control Sf9 cell lines throughout 4 different dilutions of AAV transductions.

Although the disclosed teachings have been described with reference to various applications, methods, and compositions, it will be appreciated that various changes and modifications may be made without departing from the teachings herein. The foregoing examples are provided to better illustrate the present teachings and are not intended to limit the scope of the teachings herein. Furthermore, various presently unforeseen or unanticipated alternatives, modifications, variations or improvements therein may be subsequently made by those skilled in the art which are also intended to be encompassed by the following claims. Certain aspects of the present teachings may be further understood in light of the following claims.

### REFERENCES

Chen, H. (2008) Intron splicing-mediated expression of AAV Rep and Cap genes and production of AAV vectors in insect cells. Mol Ther, 16, 924-30.
Galibert, L., A. Jacob, A. Savy, Y. Dickx, D. Bonnin, C. Lecomte, L. Rivollet, P. Sanatine, M. Boutin Fontaine, C. Le Bec & O. W. Merten (2021) Monobac System-A Single Baculovirus for the Production of rAAV. Microorganisms, 9.
Gomez-Sebastian, S., J. Lopez-Vidal & J. M. Escribano (2014) Significant productivity improvement of the baculovirus expression vector system by engineering a novel expression cassette. PLoS One, 9, e96562.
Hink, W. F. (1970) Established insect cell line from the cabbage looper, Trichoplusia ni. Nature, 226, 466-7.
Joosten, C. E. & M. L. Shuler (2003) Effect of culture conditions on the degree of sialylation of a recombinant glycoprotein expressed in insect cells. Biotechnol Prog, 19, 739-49.
Mietzsch, M., S. Grasse, C. Zurawski, S. Weger, A. Bennett, M. Agbandje-McKenna, N. Muzyczka, S. Zolotukhin & R. Heilbronn (2014) OneBac: platform for scalable and high-titer production of adeno-associated virus serotype 1-12 vectors for gene therapy. Hum Gene Ther, 25, 212-22.
Urabe, M., C. Ding & R. M. Kotin (2002) Insect cells as a factory to produce adeno-associated virus type 2 vectors. Hum Gene Ther, 13, 1935-43.
Vaughn, J. L., R. H. Goodwin, G. J. Tompkins & P. McCawley (1977) The establishment of two cell lines from the insect Spodoptera frugiperda (Lepidoptera; Noctuidae). In Vitro, 13, 213-7.

1. A genetically modified insect cell line comprising a nucleotide sequence encoding baculovirus IE1.

2. The cell line of claim 1, wherein the cell line is derived from an insect.

3. The cell line of claim 2, wherein the cell line is derived from a lepidopteran insect.

4. The cell line of claim 3, wherein the lepidopteran insect comprises Spodoptera frugiperda, Trichoplusia ni, or Bombyx mori.

5. The cell line of claim 4, wherein the cell line is derived from a Spodoptera frugiperda cell line.

6. The cell line of claim 5, wherein the cell line is derived from Spodoptera frugiperda Sf-9 designated ATCC CRL-1711.

7. The cell line of claim 4, wherein the cell line is derived from a Trichoplusia ni cell line.

8. The cell line of claim 4, wherein the cell line is derived from a Bombyx mori cell line.

9. The cell line of claim 1, wherein the cell line constitutively expresses baculovirus IE1.

10. The cell line of claim 6, wherein the cell line constitutively expresses baculovirus IE1.

11. The cell line of claim 10, wherein the cell line is characterized by a shorter doubling time relative to parental cell line Spodoptera frugiperda Sf-9 designated ATCC CRL-1711, when propagated under the same conditions.

12. The cell line of claim 10, wherein the cell line is characterized by higher AAV production yield relative to parental cell line Spodoptera frugiperda Sf-9 designated ATCC CRL-1711.

13. A method of generating a recombinant baculovirus using the cell line of claim 6.

14. A method of producing AAV vectors using the cell line of claim 6.


SEQUENCE LISTING (9 sequences)

SEQ ID NO: 1 - DNA, 40 bp, synthetic construct
  source 1..40: mol_type = other DNA
gtcgacggatcgggagatcttattaatagtaatcaattac

SEQ ID NO: 2 - DNA, 35 bp, synthetic construct
  source 1..35: mol_type = other DNA
ttgcgtcatggtggcggttggaggctggatcggtc

SEQ ID NO: 3 - DNA, 35 bp, synthetic construct
  source 1..35: mol_type = other DNA
caaccgccaccatgacgcaaattaattttaacgcg

SEQ ID NO: 4 - DNA, 60 bp, synthetic construct
  source 1..60: mol_type = other DNA
tatgatcctcgacttaattaaattcgaattttttatatttacaatttagtttttgttccg

SEQ ID NO: 5 - DNA, 35 bp, synthetic construct
  source 1..35: mol_type = other DNA
attaagtcgaggatcataatcagccataccacatt

SEQ ID NO: 6 - DNA, 40 bp, synthetic construct
  source 1..40: mol_type = other DNA
gctttttgcaaaagcctaggcttggttgttcacgatcttg

SEQ ID NO: 7 - DNA, 35 bp, synthetic construct
  source 1..35: mol_type = other DNA
attatgatcctcgacttaattaaattcgaattttt

SEQ ID NO: 8 - DNA, 20 bp, synthetic construct
  source 1..20: mol_type = other DNA
aaacctgggcagaacgtaag

SEQ ID NO: 9 - DNA, 20 bp, synthetic construct
  source 1..20: mol_type = other DNA
taagacattcatcgcgcttg